(19) United States
(12) Patent Application Publication
(10) Pub. No.: US 2008/0093529 A1
(43) Pub. Date: Apr. 24, 2008

(54) METHODS, MATERIALS, AND DEVICES FOR THE CONVERSION OF RADIATION INTO ELECTRICAL ENERGY

(76) Inventors: Mark W. Miles, San Francisco, CA (US); Damien George Hicks, Oakland, CA (US)

Correspondence Address: Hahn and Moodley LLP, P.O. Box 52050, Minneapolis, MN, 55402, US

(21) Appl. No.: 11/550,507

(22) Filed: Oct. 18, 2006

(51) Int. Cl. C12Q 1/68

(52) U.S. Cl. 250/200

(57) ABSTRACT

A rectifying nanoscale structure is disclosed, which, upon exposure to incident light, is induced to propagate electrons in an anisotropic fashion, with exceptionally low losses. A rectifying nanoscale structure which exploits the phenomena of plasmons to modify its light absorption and rectification properties is also disclosed.

### FIELD OF THE INVENTION

Embodiments of this invention relate to the methods, materials, and devices for the conversion of radiation into electricity

### BACKGROUND

A rectenna is a device that converts solar energy into electrical energy. Essentially, a rectenna comprises an optical antenna that absorbs incident solar radiation coupled to a diode that rectifies an AC voltage produced in the antenna by the incident solar radiation.

FIG. 1 shows a schematic drawing of a rectenna 100, which includes several components electrically coupled together. The rectenna 100 comprises optical antennas 102 that are integral with or connected with structures 104 that support a rectifying element 106. Bias/control circuitry 108 is located at an end of the structures 104. In use polarized light 110 is oriented so that E-field 112 is maximally coupled with the optical antennas 102 so that alternating currents are produced in the optical antennas 102 and the structures 104. Under action of the rectifying element 106, the currents are converted into a half-wave rectified output voltage. Bias/control circuitry 108 acts to apply a voltage bias to the rectifying element 106 so that its performance is maximized. The output voltage is conditioned by bias/control circuitry 108 to present minimum voltage ripple so that it is useful as a power source.

Rectennas operate on incoming radiation in the microwave frequency range which can run into the high GHz range. In the case of incoming radiation higher than the microwave frequency range, e.g. radiation in the 5-500 THz range or greater, the efficiency at which a rectenna converts incoming radiation into an output voltage is reduced. This loss in efficiency may be attributed to skin and conductor resistance effects, parasitic capacitance of the rectifying element and the coupling junctions, as well as impedance mismatch between components of the rectenna.

The above-described rectenna requires the integration of several components which include the optical antennas and the rectifying element, each of which may comprise several sub-components. The resulting geometry and interfaces between the components all contribute to the aforementioned loss in efficiency.

### SUMMARY OF THE INVENTION

According to a first aspect of the invention, there is described a rectifying nanoscale structure which, upon exposure to incident light, is induced to propagate electrons in an anisotropic fashion, with exceptionally low losses.

According to a second aspect of the invention, there is described a rectifying nanoscale structure which exploits the phenomena of plasmons to modify its light absorption and rectification properties.

According to a third aspect of the invention, there is described a rectifying nanoscale structure which exploits the phenomena of interference to enhance its efficiency.

Other aspects of the invention will be apparent from the detailed description below:

### BRIEF DESCRIPTION OF THE DRAWINGS

FIG. 1 shows a schematic drawing of rectenna known to the inventors;

FIG. 2 shows a material for solar flux conversion, in accordance with one embodiment of the invention;

FIG. 3 shows a cross section through several emitter-collector pairs, in accordance with one embodiment of the inventions;

FIG. 4a shows a side view of a material for solar flux conversion, in accordance with another embodiment of the invention;

FIG. 4b shows the material of FIG. 4a in plan view;

FIG. 5a shows a plan view of a device for solar flux conversion, in accordance with one embodiment of the invention;

FIG. 5b shows the device of 5a in packaged form;

FIG. 6 shows a solar flux conversion system, in accordance with one embodiment of the invention.

FIGS. 7a and 7b show embodiments of an edge emitter designed to produce plasmons, in accordance with one embodiment of the invention; and

FIGS. 8a and 8b show embodiments of an edge emitter coupled with an optical structure designed to improve efficiency of flux conversion, in accordance with one embodiment of the invention,

FIGS. 9a and 9b show embodiments of a collector/edge emitter array, in accordance with another embodiment of the invention.

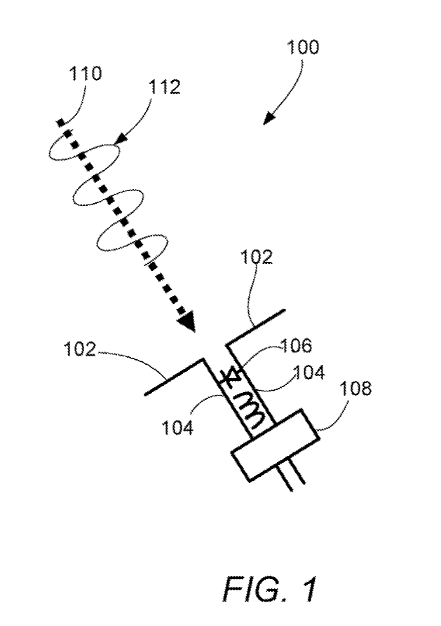

### DETAILED DESCRIPTION

In the following description, for purposes of explanation, numerous specific details are set forth in order to provide a thorough understanding of the invention. It will be apparent, however, to one skilled in the art that the invention can be practiced without these specific details.

Reference in this specification to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not other embodiments.

Embodiments of the present invention disclose a flux conversion method whereby radiation incident on a first structure results in an electron emission from the first structure towards a second structure by the process of field emission. Under the influence of the incident radiation, electrons in the first structure oscillate forming an alternating current or, more accurately, the composite of an almost infinite number of alternating currents. The geometry of the first and the second structures is such that electron field emission occurs at the first structure and not at second structure which serves to attract the electrons from the first structure. Advantageously, the first and the second structures together define a rectifier to rectify the alternating currents, without the need for a separate rectifier. Devices and materials based the flux conversion method are also disclosed.

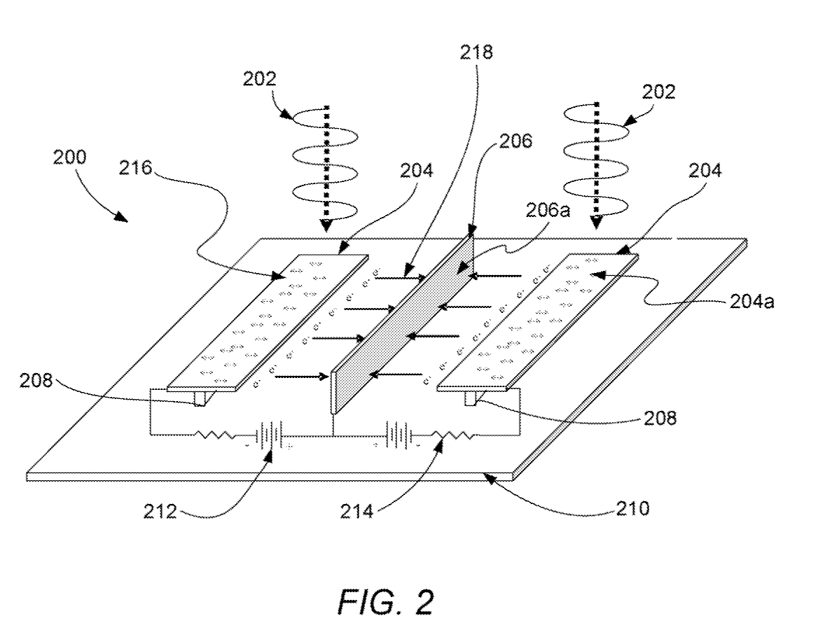

Referring now to FIG. 2, a material 200, in accordance with one embodiment of the invention, for solar flux conversion is illustrated. Incident light 202 is shown propagating toward a first structure in the form of edge emitters 204. The edge emitters 204 are positioned laterally apart and perpendicular to a second structure in the form of a collector structure 206. The edge emitters 204 are shaped and dimensioned to emit electrons responsive to the light 202 incident thereon. In one embodiment, the edge emitters 204 may comprise one or more emitter films. The emitter films may be less than several hundred nanometers thick. As will be seen, the edge emitters 204 are mounted on supports 208. The edge emitters 204 and the collector structure 206 are supported by a substrate 210. The supports 208 may be insulating. Alternatively, the supports 208 may be conducting if the substrate 210 is insulating. The supports 208 may also be opaque or transparent depending on the needs of the application. The dimensions of the supports 208 may be dependent on the characteristics of the support material of which it is comprised, and the degree to which the supports 208 and the proximity of the edge emitters 204 to the substrate 210 contribute to parasitic capacitances. In one embodiment, the supports 208 have a height less than 10 microns, and a width some fraction of the width of the edge emitters 204. The edge emitters 204 are biased by a voltage source 212, which is connected to the edge emitters 204 through current limiting resistors 214. Current limiting resistors 214 may be replaced by current limiting diodes or other semiconductor elements. In one embodiment, the incident light 202 may be polarized in which case currents 216 are induced. The currents 216 are oriented in a direction perpendicular to collectors 206. These currents are aligned with the electric field applied by the voltage source 212. With the proper bias voltage and appropriate dimensions for the edge emitters 204 and collectors 206, the currents 216 induced by the incident radiation 202 will result in electron emission in the direction indicated by arrow 218 towards the collectors 206. This will create a net direct current (DC) whose magnitude will be directly related to the intensity of the incident radiation 202. In one embodiment, the material 200 may have a number of the above-described edge emitters 204 and collectors 206 connected to a bias voltage source to convert incident radiation into a DC current.

In the material 200, the geometry of each edge emitter 204 has to be such that when coupled with the incident radiation 202 the induced electric currents 216 are sufficiently strong result in electron emission from the edge emitter by the process of field emission. In the embodiment 200, each edge emitter 204 has a body that defines as active area 204a that is operatively exposed to the incident radiation 202 and a thickness that is measured in a direction transverse to the active area 204a. In contrast to the edge emitters 204, the collector 206 is shaped and dimensioned such that any induced electric currents in the collector 204 responsive to coupling with the incident radiation 202 is too weak to result in electron emission. As will be seen, collector 206 has a collection surface 206a on which electrons from the edge emitters 204 impinge, and a thickness measured in a direction transverse to the collection surface 206a. If the aspect ratio of each edge emitter 204 is defined as the ratio of the width of the active area 204a to the thickness of the edge emitter 204, and the aspect ratio of the collector 206 is defined as the ratio of the thickness of the collector 206 to the width of the collection surface 206a, then the aspect ratio of the edge emitters 204 is higher than that aspect ratio of the collectors 206.

One skilled in the art would recognize that each edge emitter 204 is actually an antenna that is operatively coupled with incident radiation to produce free electrons, and that the combination of the edge emitter 204 with the collector 206 functions as a rectifier to rectify the alternating currents defined by the flow of free electrons within the edge emitter 204. Thus, the material 200 is a form of rectenna.

This material 200 exhibits low loss because the functions of antenna and rectifying element are structurally integrated and simple. Further, the antenna/edge emitter is a single component. Thus, parasitic capacitances are significantly reduced because there is no intervening structure. As described, the rectifying element in the form of a vacuum diode is realized in the combination of the edge emitter and the collector. The edge emitter and the collector are coupled via electrons which propagate ballistically from emitter to collector, minimizing conduction losses, and additional parasitic capacitance.

In the material 200, the geometry of each edge emitter 204 reduces the work function of the surface electrons such that incoming photons can more easily remove electrons from the edge emitter 204.

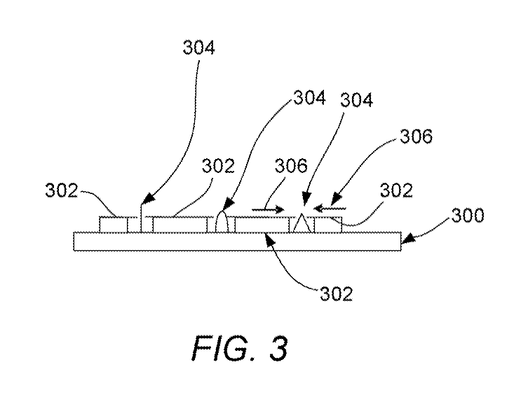

Referring now to FIG. 3, there is shown a cross-section through several emitter-collector pairs fabricated on a substrate 300, in accordance with one embodiment. As can be seen edge emitters 302 are paired with several different collector structures 304. The different collector shapes may be more easily fabricated depending on the particular manufacturing process in use. Under the influence of a similarly applied bias, carrier flow is induced along the direction indicated by arrows 306.

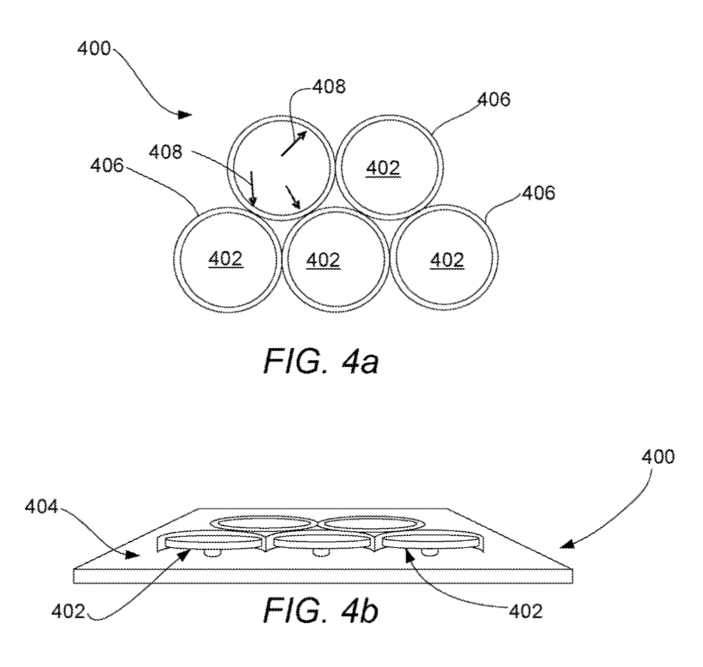

FIGS. 4a and 4b show an embodiment of a material 400 that may be used to convert radiation into electrical energy in accordance with the above-described conversion method. The material is shown in side view in FIG. 4a and in plan view in FIG. 4b. As will be seen, the material 400 comprises circular or disc-shaped edge emitters 402 fabricated on a substrate 404. The edge emitter discs 402 are encircled by collector cylinders 406. One advantage of the material 400 is it is less polarization dependent. That is to say that all of the currents induced by light which is randomly polarized can result in a net electron flow towards a collector 406 that encircles the disc 402. The electron flow is indicated by arrows 408. One skilled in the art will recognize that many other geometries are possible for emitter-collector pairs.

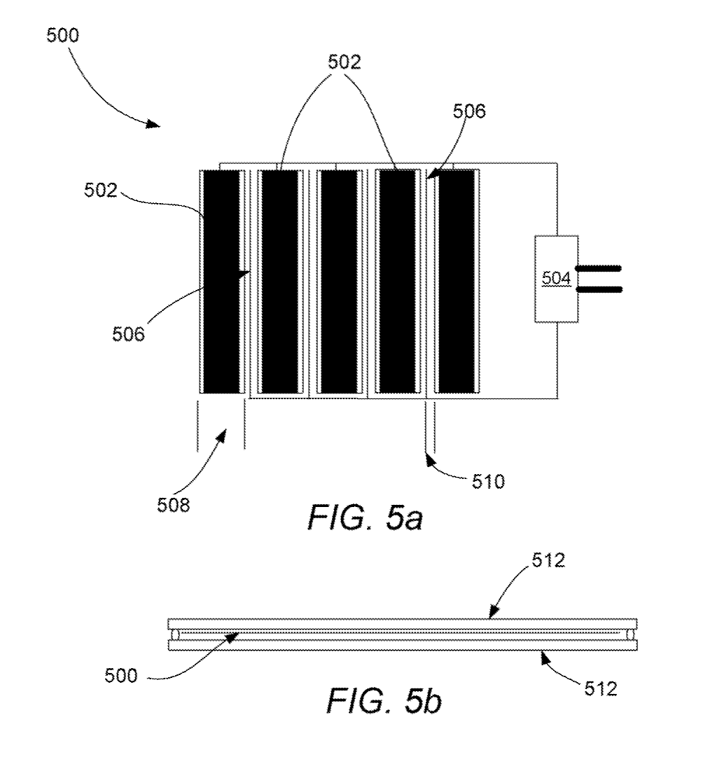

Referring to FIG. 5, a conversion device in the form of a strip array 500 comprising an array of edge emitters 502 connected in parallel to a bias/controller 504. FIG. 5a shows the strip array in plan view, whereas FIG. 5b shows the array 500 mounted in a package. For attracting electrons emitted from the edge emitters 502, the device 500 comprises a plurality of collectors 506. Each edge emitter 502 as a lateral dimension 508 which may be bounded approximately on the upper end by the transverse spatial coherence of the incident radiation. One possible mechanism for bounding the lower end relies on classical antenna theory.

The transverse spatial coherence length for direct sunlight is given by L=0.16Rλ/ρ, where R is the distance to the sun (1.5×10¹¹ m) and ρ is the sun's radius (7×10⁸ m). For optical wavelengths this is greater than 10 microns, so the lateral dimension 508 is less than 10 microns for the incident light electric field across an emitter 502 to be in phase. Optimum efficiency for a half-wave dipole antenna requires the antenna dimension in the wave oscillation direction to be integer multiples of ½ the wavelength of the incident light. With the shortest wavelength contained in solar radiation being about 200 nanometers, this corresponds to an emitter 502 dimension of greater than 100 nanometers.

The separation 510 between edge emitters 502 and collectors 506 may be constrained by a combination of required diode behavior, manufacturing capabilities, and the required bias voltage, in one embodiment. In one embodiment, the separation 510 may range from 0.05 microns to 1 microns.

Bias/controller 504 serves to provide a bias voltage between the edge emitters and collectors to increase the conversion efficiency of the embodiment 500. A variety of factors determine the bias voltage, including the aforementioned dimensions, properties of the materials comprising the structures, and the strength of the solar flux. In one embodiment the bias voltage may be adaptively changed based on changes in the solar flux strength due to the time of day and weather conditions.

Referring now to FIG. 5b, the array 500 is shown in a vacuum enclosure/package defined by layers 512 where one of the layers must be transparent. Depending on the separation 510 between the emitter 502 and collectors 506, in some embodiments it may not be necessary to package the device in a vacuum. If the separation 510 is sufficiently small then carrier transport may occur without any significant effects due to scattering. For distances of 1 micron or less, electron transport at atmospheric pressures is essentially collisionless.

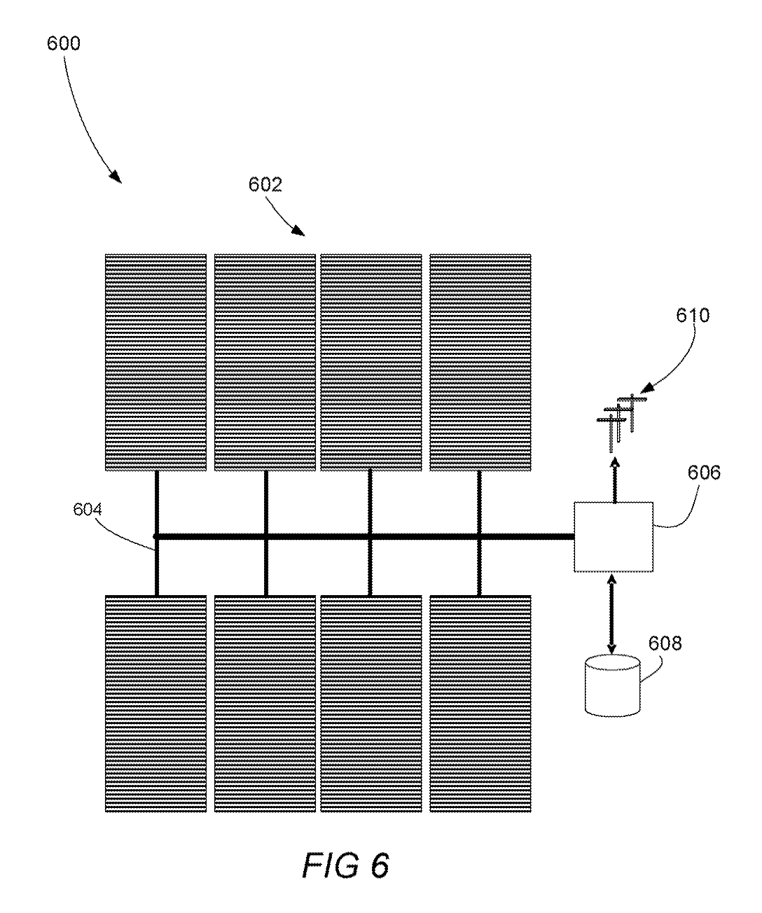

Referring now to FIG. 6, a solar flux conversion system 600, in accordance with one embodiment of the invention is shown. The system 600 comprises an array 602 of flux conversion devices as described above. Each of the devices in the array 602 may be connected in a serial and/or parallel via a bus 604 to a bias/controller element 606. The bias/controller element 606 performs bias functions and optimizes conversion efficiency. The bias/controller element 606 also controls the charging of an energy storage unit 608, as well as the inversion (conversion from DC to AC) and distribution of energy to an electrical grid 610. The system 600 can act as a self-contained energy generation node that is part of a larger energy generation and distribution network. Each node may be capable of satisfying some or all the needs of a local user or host, and then intelligently supplying excess power to an existing electrical distribution grid.

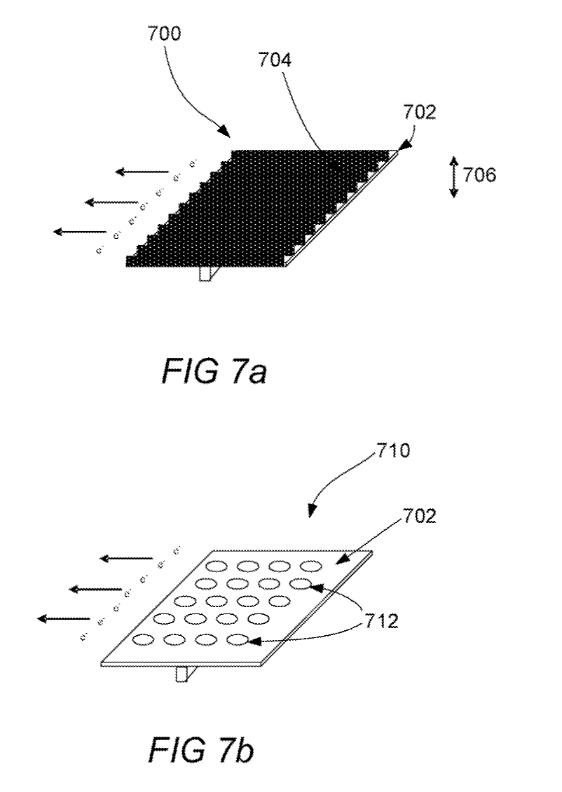

Referring now to FIG. 7a, there is shown a schematic drawing of an edge emitter 700, in accordance with one embodiment of the invention. The edge emitter is similar to the edge emitter 204 described above, but includes an active area 702 that has a morphology. In other words, the active area 702 has surface features or periodic structures 704 which introduce variations in height along the axis illustrated by arrow 706. These features may reside on the top surface., the bottom surface, or both. These features may be of any geometry or arrangement. In general, however, their dimensions vary from the sub-micron to sub-nanometer scale.

FIG. 7b shows an embodiment 710 of an edge emitter in accordance with one embodiment of the invention. The embodiment 710 is very similar to the embodiment 700 except that the active area 702 includes discontinuities. In the embodiment shown the discontinuities are in the form of apertures 712. The apertures 712 may be of arbitrary geometry and arrangement and may be in the sub-micron to sub-nanometer size range

The surface features of edge emitters 700 and 710 exploit the phenomena of plasmons to enhance overall performance. The theory of plasmons is described in “A Hybridization Model for the Plasmon Response of Complex Nanostructures,”, N., Halas E. Prodan et. al., Journal of Science, Oct. 17, 2003. Plasmons can be described as electron density waves which propagate on a metal surface. The specific nature of the plasmon is related to the geometry of the surface which accommodates it. The phenomenon is of use because plasmons can produce and alter the nature of the electric fields generated when light is incident on a metallic structure, as well as the manner in which light is absorbed and/or converted into electric fields. The incorporation of sub-micron and sub-nanometer structures into the edge emitter disclosed herein provides an additional mechanism by which the electron emission and light conversion and absorption properties of edge emitters may be manipulated.

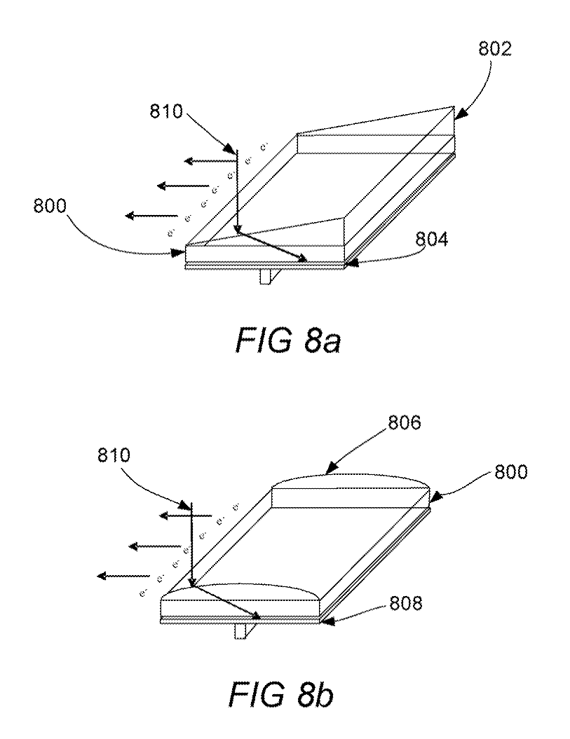

In some embodiments the edge emitter may be coupled with an optical structure to increase an angle of incidence at which radiation strikes the active area of the edge emitter. FIG. 8a shows an edge emitter 800 which is similar to any of the above described edge emitters to which is coupled an optical structure in the form of a prism 802. The prism 802 may be coupled to the active area of the edge emitter 800 via at least one coupling film 804. FIG. 8b shows an embodiment in which the edge emitter 800 is coupled to an optical structure in the form of a hemispherical lens 806 via at least one coupling film 808.

The optical structures 804 and 806 serve to take light 810 which is incident normally, and increase the angle of incidence with which it strikes the active area to which it is coupled. Light which is incident on a surface at higher angles of incidence has enhanced ability to induce surface plasmons. Consequently the efficiency of this effect can be increased by proper design of the optical structure. These structures may take on arbitrary shapes depending on the expected average angle of incidence on the structure as a whole during operation. The structure is generally made from a material which is transparent at the wavelengths of interest. In addition to optical structure designs based on refraction, designs exploiting the phenomena of diffraction and interference are also useful and may present design advantages.

The coupling films 804 and 808 are made of materials that are also optically transparent to allow radiation to pass thorough to the edge emitter on which it resides. The thickness and refractive properties of these films are chosen to enhance coupling of the light into the surface. In general this requires that at least one of the films be of a material with a refractive index less than that of the optical structure. One of the films may also be air.

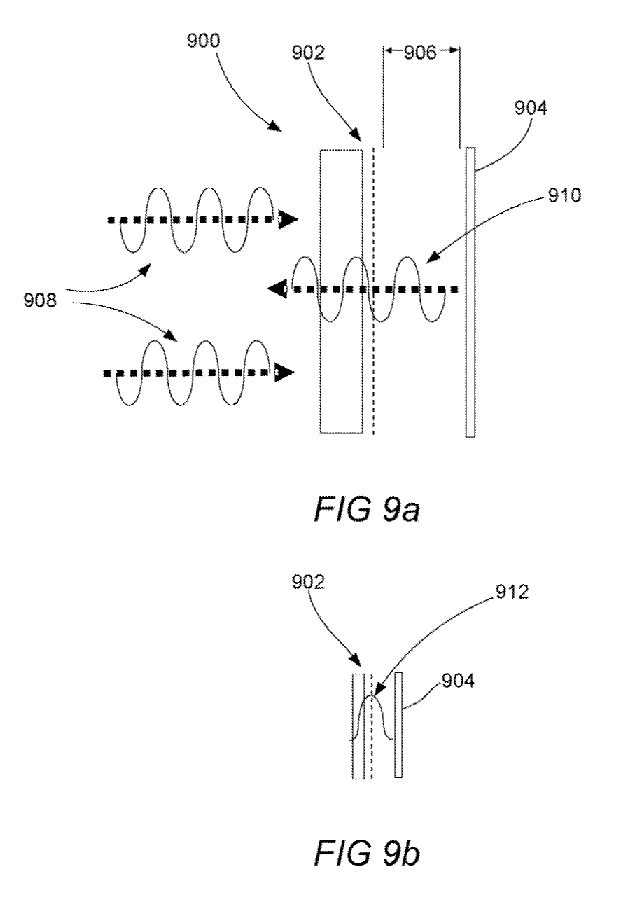

Referring now to FIG. 9a, a rectenna 900 is shown, in accordance with one embodiment of the invention. The rectenna 900 is designed to ensure greater conversion of energy from incident light. The rectenna 900 comprises a transparent collector-emitter structure indicated generally by reference numeral 902. The collector-emitter structure 902 includes an emitter and a collector as described above. The collector-emitter structure 902 is laterally spaced from a reflecting substrate 904 by a gap 906. Radiation 908 reaches the emitter of the collector-emitter structure 902 and causes an alternating current to flow in a body of the emitter. This alternating current is rectified in a process in which electrons are emitted from the emitter body and made to flow towards the collector of the collector-emitter structure 902. The reflecting substrate 904 produces a standing wave upon reflection due to interference between the radiation 908 and a reflected wave 910. In one embodiment, the peak of the standing wave may be positioned to coincide with the location of the collector-emitter structure 902 by fixing the gap 906 appropriately. The gap 906 may be fixed at less than one micron for visible light. If the peak of the standing wave is coincident with the collector-emitter structure 902 then an increased field strength results which can enhance the emission of electrons from the emitter body. FIG. 9b illustrates a standing wave peak 912 produced by reflection by the substrate 904. As will be seen, peak 912 is coincident or aligned with collector-emitter structure 902.

In the embodiments of the flux conversion device described above, radiation in the form of solar energy is converted into electrical energy. However, one skilled in the art would appreciate that the techniques and devices disclosed herein are suitable for the conversion of energy from other regions of the electromagnetic spectrum into electrical energy.

While certain exemplary embodiments have been described and shown in the accompanying drawings, it is to be understood that such embodiments are merely illustrative and not restrictive of the broad invention and that this invention is not limited to the specific constructions and arrangements shown and described, since various other modifications may occur to those ordinarily skilled in the art upon studying this disclosure. In an area of technology such as this, where growth is fast and further advancements are not easily foreseen, the disclosed embodiments may be readily modifiable in arrangement and detail as facilitated by enabling technological advancements without departing from the principals of the present disclosure or the scope of the accompanying claims.

What is claimed is:

1. A material, comprising:
a first structure shaped and dimensioned to support an induced alternating current responsive to radiation incident thereon; and
a second structure spatially separated from the first structure by a non-solid medium;
wherein the first and the second structures together functioning as a rectifier to rectify the induced alternating current in a process where electrons are emitted from the first structure and tend to travel through the non-solid medium towards the second structure, the second structure being shaped and dimensioned to attract the electrons.

2. The material of claim 1, wherein the first structure is oriented in an operative position to increase its coupling with the incident radiation, whereas the second structure is oriented in the operative position to decrease tits coupling with the incident.

3. The material of claim 1, wherein the first structure has a low work function

4. The material of claim 1, wherein the first structure has a body that defines an active area that is operatively exposed to the incident radiation and a thickness measured in a direction transverse to the active area.

5. The material of claim 1, wherein that second structure has a body that defines a collection surface on which the electrons from the first structure impinge and a thickness measured in a direction transverse to the collection surface.

6. The material of claim 5, wherein an aspect ratio of the first structure is higher than an aspect ratio of the second structure, wherein the aspect ratio of the first structure is defined as the ratio of the width of the active area to the thickness of the first structure, and the aspect ratio of the second structure is defined as the ratio of thickness of the second structure to the width of the collection surface.

7. The material of claim 5, wherein the collection surface has a morphology to permit coupling with the incident radiation to induce surface plasmons on the collection surface.

8. The material of claim 7, wherein the morphology is such that there are variations in the thickness of the body of the first structure on the sub-micron to sub-nanometer range.

9. The material of claim 7, wherein the morphology is due to sub-micron to sub-nanometer scale structures in the active area.

10. The material of claim 7, wherein the morphology is due to sub-micron to sub-nanometer scale discontinuities in the active area.

11. The material of claim 4, wherein the active area is coupled to an optical structure which increases an angle of incidence at which radiation strikes the active area.

12. The material of claim 11, wherein the optical structure is coupled to the active area via at least one coupling film disposed between the filter and the active area.

13. The material of claim 12, wherein the coupling film has a refractive index less that that of the material of the optical structure.

14. The material of claim 11, wherein the optical structure comprises a transparent prism.

15. The material of claim 11, wherein the optical structure comprises a hemispherical lens.

16. The material of claim 1, further comprising a reflector to reflect radiation passing through the first and second structures back towards the first and second structures.

17. The material of claim 16, wherein the reflector is separated from the first and second structures by a gap selected to cause the radiation reflected from the reflector to interfere with radiation incident on the reflector thereby to create a standing wave having a peak spatially located between the first and the second structures.

18. A rectenna, comprising;
an antenna realized by an emitter element capable of being coupled with incident radiation to produce an alternating current in a body of the emitter element; and
a rectifier realized by a combination of the emitter element and a collector element spatially separated from the emitter element by a non-solid medium to rectify the alternating current in a process wherein electrons are emitted from the emitter element and attracted towards the collector element.

19. The rectenna of claim 18, wherein the emitter element has an active area which is operatively exposed to the incident radiation and a thickness measured in a direction that is transverse to the active area.

20. The rectenna of claim 18, wherein that collector element has a collector area that is operatively impinged by electrons flowing from that emitter element, and a thickness that is measured in a direction that is transverse to the collection area.

21. The rectenna of claim 20, wherein an aspect ratio of the emitter element is higher than an aspect ratio of the collector element, the aspect ratio of the emitter element being defined as the ratio of a width of the active area to the thickness of the emitter element, and the aspect ratio of the collector element being defined as a ratio of the thickness of the collection element to the width of the collection area.

22. The rectenna of claim 19, wherein the active area has a morphology to induce surface plasmons in the active area when struck by incident radiation.

23. The rectenna of claim 22, wherein the morphology comprises structures on the active area that vary its thickness.

24. The rectenna of claim 22, wherein the morphology comprises discontinuities in the active area.

25. The rectenna of claim 19, further comprising an optical structure coupled to the active area to increase an angle of incidence at which radiation is incident on the active area.

26. The rectenna of claim 25, wherein the optical structure is coupled to the active area via at least one coupling film disposed between the filter and the active area.

27. The rectenna of claim 26, wherein the coupling film as a refractive index less that that of the optical structure.

28. The rectenna of claim 25, wherein the optical structure comprises a transparent prism.

29. The rectenna of claim 25, wherein the optical structure comprises a hemispherical lens.

30. The rectenna of claim 18, further comprising a reflector to reflect radiation passing through the antenna back into the antenna, the reflector being at a distance from the antenna to generate a standing wave through interference between the incident radiation and the reflected radiation whose peak is spatially located between the emitter element and the collector element.

31. A method, comprising:
coupling incident radiation an emitter element to produce an alternating current in the emitter element; and
rectifying the alternating current in a a process in which electrons are emitted from the emitter element and made to flow towards a collector element which is separated from the emitter element by a non-solid medium.

32. The method of claim 31, wherein coupling the incident radiation with the emitter element comprises increasing an angle of incidence at which the incident radiation strikes the emitter element.

33. The method of claim 32, wherein increasing the angle of incidence comprises passing the incident radiation through a hemispherical lens before it strikes the emitter element.

34. The method of claim 32, wherein increasing the angle of incidence comprises passing the incident radiation through a prism before it strikes the emitter element.

35. The method of claim 31, wherein coupling that incident radiation with the emitter element comprises inducing surface plasmons on a surface of the emitter element

36. The method of claim 31 wherein inducing the surface plasmons comprises introducing variations in a thickness of the emitter element measured in a direction transverse to the surface.

37. The method of claim 36, wherein the variations in the thickness is in the sub-micron to sub-nanoscale range.

38. The method of claim 31, wherein inducing the surface plasmons comprises introducing discontinuities in the surface.

39. The method of claim 31, further comprising applying a bias voltage between the emitter element and the collector element to induce electron flow form the emitter element to the collector element.

40. The method of claim 31, further comprising generating a standing wave having a peak spatially located between the emitter element and the collector element.

41. The method of claim 40, wherein generating the standing wave comprises reflecting radiation passing through the emitter back in the direction of the emitter by a reflector.

42. A rectenna, comprising:
a collector element having a body shaped and dimensioned to attract electrons;
an emitter element having a body shaped and dimensioned to interact with incident radiation to produce an alternating current within the body, wherein the alternating current is rectified in a process in which electrons are emitted from the emitter element and made to flow towards the collector element.

43. A material, comprising:
an emitter; and
a collector capable of being coupled to the emitter via electrons that propagate from the emitter to the collector in response to radiation incident on the emitter, wherein the emitter is shaped and dimensioned to increase its coupling with the incident radiation and the collector is shaped and dimensioned to reduce its coupling with the incident radiation

44. A material, comprising:
an emitter structure; and
a collector structure spatially separated from the emitter structure, the emitter and the collector structures being shaped and dimensioned to in cooperation set up an anisotropic current flow from the emitter structure to the collector structure in response to radiation incident to the emitter structure.

45. A material, comprising:
an antenna which can be coupled with incident radiation to produce an alternating current in a body of the antenna, and
a rectifying mechanism to rectify the alternating current in a process comprising an asymmetric transfer of electrons from the body of the antenna through a non-solid medium.
